(19) United States
(12) Patent Application Publication
(10) Pub. No.: US 2010/0056430 A1
(43) Pub. Date: Mar. 4, 2010

(54) Treatment and Method for Eliminating or Reducing Foot Odor

(76) Inventor: Arnold Lester, Burleson, TX (US)

Correspondence Address: John T. Wilson, Esq., Suite 2500, 16660 Dallas Parkway, Dallas, TX, 75248, US

(21) Appl. No.: 12/199,088

(22) Filed: Aug. 27, 2008

(51) Int. Cl. A61K 38/12

(52) U.S. Cl. 514/9

(57) ABSTRACT

A method of treating a person having a foot odor comprises administering an effective foot odor treatment amount of an antimicrobial preparation, such as Polymyxin B. Sulfate, Bacitracin Zinc, and an antimicrobial such as Miconazole Nitrate, terbinafine hydrochloride, undecylenic acid, tolnaftate, clotrimazole, or butenafine hydrochloride, to eliminate or reduce the symptoms of foot odor.

The present invention is a topical skin preparation for treatment of foot odor. The preparation comprises Polymyxin B Sulfate and Bacitracin Zinc in combination with an antimicrobial agent. In a preferred form, the preparation further comprises, an antimicrobial such as Miconazole Nitrate. The concentrations of these constituents are 10,000 units of Polymyxin B Sulfate, 500 units of Bacitracin Zinc and 1.0 to 2.0% concentration of Miconazole Nitrate, in one preferred embodiment. Other compounds, such as herbal and fragrance additives may also be added to the preparation, for example Rose Water, Colors, Vitamin E, Aloe, Vitamin C, and Collagen. Further, other antimicrobial agents such as terbinafine hydrochloride, undecylenic acid, tolnaftate, clotrimazole, butenafine hydrochloride may be substituted for the antimicrobial agent Miconazole Nitrate.

The topical skin preparation may further include Topical Erythromycin, Topical Aluminum Chloride Hexahydrate, or Tea Tree Oil. Topical Erythromycin may be used as a gel or solution with a 2.0% concentration. Topical Aluminum Chloride Hexahydrate may be used in a concentration of about 5.0% to 20.0%. Tea Tree Oil may be used in a concentration of about 0.10% to 10%.

### CROSS-REFERENCE TO RELATED APPLICATIONS

None.

### TECHNICAL FIELD

The present invention is generally a topical skin treatment. More specifically, the present invention is a topical skin treatment and method effective at eliminating or reducing foot odor.

### BACKGROUND

Foot odor is a type of body odor that affects the feet of humans and is generally considered to be an unpleasant smell. Since human feet are densely covered with ecrine sweat glands, excessive perspiration of the feet is the result where inadequate air ventilation exists. Foot odor may result from wearing shoes and/or socks, especially shoes or socks which are moist or remain on the body for several hours. Hair on the feet, especially on the toes, may also add to odor intensity by adding increased surface area for the bacteria to thrive in. Since socks absorb varying amounts of perspiration from feet, wearing shoes without socks may increase the amount of perspiration contacting feet and increase odor causing bacterial activities. Women wearing nylon stockings or pantyhose may also experience increased foot odor.

The quality of foot odor is often reported as a thick, cheesy smell, which may be described as a smell equivalent to malt vinegar or ammonia-like. Foot odor can sometimes be caused by wearing the same socks too many times in a row and sweating. Brevibacteria and certain types of gram positive/gram negative bacteria are considered a major cause of foot odor, as they ingest dead skin on the feet, especially on the soles and between the toes, converting in the process the amino acid methionine to methanethiol which has a sulfuric aroma. The brevibacteria gives cheeses such as Limburger, Bel Paese, Muenster, and the like, their pungency.

In addition, propionic acid (propanoic acid) of foot sweat, results in Propionibacteria which thrives in the ducts of adolescent and adult sebaceous glands. As propionic acid is chemically structured similarly to acetic acid producing similar physical characteristics including odors, this may account for the foot odors identified as being vinegar-like smell by certain persons. Isovaleric acid (3-methyl butanoic acid) is the other source of foot odor as a result of actions of the bacteria Staphylococcus epidermidis, which is also present in several strong cheese types.

Among the earliest foot deodorants were aromatic herbs such as allspice, which nineteenth-century Russian soldiers would put in their boots. Some types of powders and activated charcoal insoles, such as Odor Eaters, have been developed to combat foot odor by keeping the feet dry.

### SUMMARY

A method of treating a person having a foot odor comprises administering an effective foot odor treatment amount of an antimicrobial preparation, such as Polymyxin B. Sulfate, Bacitracin Zinc, and an antimicrobial such as Miconazole Nitrate, terbinafine hydrochloride, undecylenic acid, tolnaftate, clotrimazole, or butenafine hydrochloride, to eliminate or reduce the symptoms of foot odor.

The present invention is a topical skin preparation for treatment of foot odor. The preparation comprises Polymyxin B Sulfate and Bacitracin Zinc in combination with an antimicrobial agent. In a preferred form, the preparation further comprises, an antimicrobial such as Miconazole Nitrate. The concentrations of these constituents are 10,000 units of Polymyxin B Sulfate, 500 units of Bacitracin Zinc and 1.0 to 2.0% concentration of Miconazole Nitrate, in one preferred embodiment. Other compounds, such as herbal and fragrance additives may also be added to the preparation, for example Rose Water, Colors, Vitamin E, Aloe, Vitamin C, and Collagen. Further, other antimicrobial agents such as terbinafine hydrochloride, undecylenic acid, tolnaftate, clotrimazole, butenafine hydrochloride may be substituted for the antimicrobial agent Miconazole Nitrate.

The topical skin preparation may further include Topical Erythromycin, Topical Aluminum Chloride Hexahydrate, or Tea Tree Oil. Topical Erythromycin may be used as a gel or solution with a 2.0% concentration. Topical Aluminum Chloride Hexahydrate may be used in a concentration of about 5.0% to 20.0%. Tea Tree Oil may be used in a concentration of about 0.10% to 10%.

### BRIEF DESCRIPTION OF THE DRAWINGS

None.

What is claimed is:

1. A topical antimicrobial preparation comprising: A therapeutically effective concentration of polymyxin B sulfate and bacitracin zinc in order to act against bacteria on skin to reduce odor; and, an antimicrobial agent.

2. A topical antimicrobial preparation as recited in claim 1, wherein said antimicrobial agent is miconazole nitrate.

3. A topical antimicrobial preparation as recited in claim 1, wherein said preparation comprises about 5,000 to 10,000 units of polymyxin B sulfate, about 400 to 500 units of bacitracin zinc, and about 1 to 2 percent concentration miconazole nitrate.

4. A topical antimicrobial preparation as recited in claim 1, wherein said antimicrobial agent is selected from the group consisting of terbinafine hydrochloride, undecylenic acid, tolnaftate, clotrimazole, or butenafine hydrochloride.

5. A topical antimicrobial preparation as recited in claim 4, wherein said preparation comprises about 5,000 to 10,000 units of polymyxin B sulfate, about 400 to 500 units of bacitracin zinc, and about 0.75 to 1 percent concentration terbinafine hydrochloride.

6. A topical antimicrobial preparation as recited in claim 4, wherein said preparation comprises about 5,000 to 10,000 units of polymyxin B sulfate, about 400 to 500 units of bacitracin zinc, and about 10 to 20 percent concentration undecylenic acid.

7. A topical antimicrobial preparation as recited in claim 4, wherein said preparation comprises about 5,000 to 10,000 units of polymyxin B sulfate, about 400 to 500 units of bacitracin zinc, and about 0.50 to 1 percent concentration tolnaftate.

8. A topical antimicrobial preparation as recited in claim 4, wherein said preparation comprises about 5,000 to 10,000 units of polymyxin B sulfate, about 400 to 500 units of bacitracin zinc, and about 0.50 to 1 percent concentration clotrimazole.

9. A topical antimicrobial preparation as recited in claim 4, wherein said preparation comprises about 5,000 to 10,000 units of polymyxin B sulfate, about 400 to 500 units of bacitracin zinc, and about 0.50 to 1 percent concentration butenafine hydrochloride.

10. A topical antimicrobial preparation as recited in claim 4, wherein said preparation further comprises an agent selected from the group consisting of topical erythromycin, topical aluminum chloride hexahydrate, or tea tree oil.

11. A topical antimicrobial preparation as recited in claim 10, wherein said preparation comprises about 2.0% to 5.0% topical erythromycin.

12. A topical antimicrobial preparation as recited in claim 10, wherein said preparation comprises about 5.0% to 20.0% topical aluminum chloride hexahydrate.

13. A topical antimicrobial preparation as recited in claim 10, wherein said preparation comprises about 0.10% to 10.0% tea tree oil.

14. A method of treating a person having a foot odor, comprising topically administering an effective treatment amount of an antobotic preparation to said person to eliminate or reduce the symptoms of said foot odor; wherein said foot odor is cause by bacteria, fungi, and yeast.

15. The method of claim 14, wherein said antimicrobial preparation is administered at least once a day in an amount of about 1/50^{th} of gram to 1 gram, as required by foot area size being treated.

16. The method according to claim 15, wherein said antimicrobial preparation is applied in a vehicle selected from the group consisting of aqueous liquid, non-aqueous liquid, salve, ointment, cream, and powdered solid excipient.

17. The method of claim 16, wherein said antimicrobial preparation comprises about 5,000 to 10,000 units of polymyxin B sulfate, about 400 to 500 units of bacitracin, and about 1 to 2 percent concentration miconazole nitrate.

18. The method of claim 16, wherein said antimicrobial agent is selected from the group consisting of terbinafine hydrochloride, undecylenic acid, tolnaftate, clotrimazole, or butenafine hydrochloride.

19. The method of claim 16 wherein said antobotic preparation comprises about 5,000 to 10,000 units of polymyxin B sulfate, about 400 to 500 units of bacitracin, and about 0.75 to 1 percent concentration terbinafine hydrochloride.

20. The method of claim 16 wherein said antobotic preparation comprises about 5,000 to 10,000 units of polymyxin B sulfate, about 400 to 500 units of bacitracin, and about 10 to 20 percent concentration undecylenic acid.

21. The method of claim 16, wherein said antobotic preparation comprises about 5,000 to 10,000 units of polymyxin B sulfate, about 400 to 500 units of bacitracin, and about 0.50 to 1 percent concentration tolnaftate.

22. The method of claim 16, wherein said antobotic preparation comprises about 5,000 to 10,000 units of polymyxin B sulfate, about 400 to 500 units of bacitracin, and about 0.50 to 1 percent concentration clotrimazole.

23. The method of claim 16, wherein said antobotic preparation comprises about 5,000 to 10,000 units of polymyxin B sulfate, about 400 to 500 units of bacitracin, and about 0.50 to 1 percent concentration butenafine hydrochloride.

24. The method of claim 16, wherein said antobotic preparation, wherein said preparation further comprises an agent selected from the group consisting of topical erythromycin, topical aluminum chloride hexahydrate, or tea tree oil.

25. The method of claim 16, wherein said preparation comprises about 2.0% to 5.0% topical erythromycin.

26. The method of claim 16, wherein said preparation comprises about 5.0% to 20.0% topical aluminum chloride hexahydrate.

27. The method of claim 16, wherein said preparation comprises about 0.10% to 10.0% tea tree oil.
